(19) United States
(12) Patent Application Publication
(10) Pub. No.: US 2022/0125686 A1
(43) Pub. Date: Apr. 28, 2022

(54) TITANIUM-CONTAINING SILICATE-BASED BIOACTIVE GLASS FOR ENAMEL REMINERALIZATION

(71) Applicant: THE ROYAL INSTITUTION FOR THE ADVANCEMENT OF LEARNING/MCGILL UNIVERSITY, Montreal (CA)

(72) Inventors: Marta CERRUTI, Montreal (CA); Sophia SMITH, Cambridge, MA (US); Gabriele CAPILLI, Montreal (CA); Faleh Ahmad TAMIMI MARINO, Montreal (CA); Chisokwuo Perpetual Akunna, Montreal (CA)

(73) Assignee: THE ROYAL INSTITUTION FOR THE ADVANCEMENT OF LEARNING/MCGILL UNIVERSITY

(21) Appl. No.: 17/507,842

(22) Filed: Oct. 22, 2021

Related U.S. Application Data
(60) Provisional application No. 63/104,832, filed on Oct. 23, 2020.

(51) Int. Cl. A61K 6/20; A61K 6/77; A61K 6/71; A61K 6/76

(52) U.S. Cl. CPC A61K 6/20; A61K 6/76; A61K 6/71; A61K 6/77

(57) ABSTRACT

A bioactive glass composition comprising titanium homogenously incorporated and distributed throughout the glass and method of making a sol-gel or melt-derived bioactive glass, the bioactive glass increasing osteogenic differentiation and can be used for enamel remineralization in dentistry or bone tissue engineering.

### CROSS-REFERENCE TO RELATED APPLICATIONS

The present application claims benefit of U.S. Provisional Application No. 63/104,832 filed Oct. 23, 2020, the content of which is hereby incorporated by reference in its entirety.

### TECHNICAL FIELD

It is provided a titanium containing bioactive glass and method of making same.

### BACKGROUND

Enamel is the hardest and most heavily mineralized material in the human body. It is composed of 96% mineral (hydroxyapatite), 4% organic matter and a variety of trace metallic elements such as selenium, chromium and titanium. Titanium (Ti) presence has been correlated with increased tooth hardness and brightness, both sought after attributes for clinical applications.

The enamel layer is susceptible to demineralization and is unable to fully remineralize by biological methods as it is not living tissue. As the tooth demineralizes, issues arise in overall tooth health, ranging in severity from dental sensitivity to the formation of cavities and eventual potential tooth loss, all effecting that most human of attributes—the smile. There are several methods to remineralize enamel, one of which is bioactive glass.

Bioactive glass applications range from the repair of defects in hard tissue to enamel remineralization with great commercial success. This material is reactive when placed in physiological fluid, undergoing a transformation from glass to hydroxyapatite (HA) or hydroxy carbonated apatite, the main mineral component of enamel. The formation of a HA layer and the ability to release ions results in osteo-conductive and osteo-stimulative properties that sets bioactive glass apart. Synthesis is achieved by a melt-quenched process or a sol-gel process. Sol gel processing allows for glass with more variable composition and higher surface area than melt derived processing routes.

Bioactive glass networks also support the possibility of ionic doping. Numerous metallic oxides have been incorporated as network modifiers, resulting in modifications to the physical and chemical properties of the glass. The majority of the metals incorporated are released to the local physiological environment, generally for targeted antibacterial applications. The release rate of ions is controlled by glass dissolution rate in conjunction with the rate of tissue healing.

Release of ions such as copper, silver and cobalt from bioactive glasses have been extensively explored. The inclusion of Ti ions has been far less researched and especially the accumulation of Ti at the bioglass surface upon its immersion in physiological fluids. However, several studies have looked at Ti-containing glasses of various compositions and processing methods. Using primarily the melt-derived method, Ti-containing bioactive glasses have been made based on phosphate, silicate, and lithium borosilicate glass networks where concentration of Ti above 20 mol % resulted in significant crystallization of the glass network.

Rodriguez et al. (2017, Heliyon 3(10) (2017) e00420-e00420) examined Ti containing silicate-based melt-derived bioactive glasses for the purposes of antibacterial coatings on metallic implants. The introduction of Ti did not significantly alter the solubility behavior up to 15 mol % Ti. However, the inclusion of Ti did result in some degree of crystallinity. Ti was consistently released to solution throughout immersion in water, approximately 5 ppm after 7 days of immersion. This glass also induced a decrease in osteoblast cell proliferation as a result of contact. Ni et al synthesized sol-gel derived bioactive glass 58S with Ti inclusions, based on a silicate network with additions of CaO, P₂O₅ and Na₂O. In this case, the introduction of Ti did not result in major crystallinity. However, it did effect the dissolution behavior of the glass, slowing it down compared to non-Ti containing glasses of the same base composition.

Another issue with high concentration of Ti in the glass network is that it tends to slow down the glass dissolution and therefore decrease bioactivity (i.e. formation of hydroxyapatite upon contact with body fluids). Thus increasing the concentration of Ti in bioactive glass networks is an unresolved issue.

These difficulties and drawbacks in current Ti-containing bioactive glasses illustrate the need for further exploration of Ti inclusion, as if improved, Ti-containing bioactive glass could potentially have powerful effects in a variety of applications. While research on Ti-inclusion in bioactive glass is lacking, inclusion of Ti in HA has shown superior properties such as better mechanical, antibacterial, cellular and aesthetic improvement when compared to unmodified HA. In the case of Ti inclusion in HA, Ti is incorporated in a way that allows for Ti retention in the material, rather than release to the local environment. Existing bioactive glasses with Ti inclusions are specifically designed for Ti release throughout the process of transformation to HA.

Thus there is a need to be provided with new means to incorporate titanium in material such as bioactive glass in such a way that Ti is incorporated homogenously in the glass and is retained at the glass surface once the glass is in contact with physiological solution. There is also a need for a Ti-containing bioactive glass that reacts fast enough upon contact with body fluids.

### SUMMARY

It is provided a method of preparing a titanium-containing bioactive glass comprising the steps of incorporating titanium (Ti) to silica producing a mixture, mixing said mixture containing titanium to high viscosity producing a sol, casting the sol in a vial and gelling said sol resulting a gel, and calcine said gel at high temperature producing the titanium-containing bioactive glass.

It is further provided a method of preparing a melt-quenched titanium-containing bioactive glass comprising the steps of mixing and homogenizing a powder mixture comprising silica and titanium (Ti) producing a powder mixture, melting the powder mixture at high temperature generating a melt, and quenching the melt at low temperature resulting in melt-quenched titanium-containing bioactive glass.

It is also provided a bioactive glass composition comprising titanium (Ti) homogenously incorporated and distributed throughout the glass, wherein the Ti is retained at the glass surface once the glass is in contact with a physiological solution and is incorporated within an hydroxyapatite.

In an embodiment, the gel is further aged at 37° C. for 48 hrs prior to be calcined.

In another embodiment, the aged gel is further air dried at room temperature for further 24 hours prior to be calcined.

In an alternate embodiment, the silica and titanium are mixed producing a powder mixture, said powder mixture being further melted and quenched at low temperature resulting in melt-quenched titanium-containing bioactive glass.

In an embodiment, the titanium is incorporated using an acid catalyzer or a basic catalyzer.

In an embodiment, the acid catalyzer is HCl or HNO₃, or the basic catalyzer is NaOH, Na₂CO₃, NH₄OH or CaOH₂. Preferably, the basic catalyzer is CaOH₂.

In a supplemental embodiment, the gel is calcined at 700° C.

In an embodiment, the titanium-containing bioactive glass is further ground, sieved or polished.

In another embodiment, the powders are mixed and ground in a mortar.

In a further embodiment, the powder mixture is melted at a high temperature between 1000 and 1500° C.

In another embodiment, the powder mixture is melted in a muffle furnace.

In an embodiment, the powder mixture is placed in a platinum crucible before melting.

In a supplemental embodiment, further comprising incorporating calcium, magnesium, sodium, phosphate, potassium, lithium, or a combination thereof to the mixture to Ti and silica.

In an embodiment, the silica is in the form of tetraethyl orthosilicate (TEOS), tetramethylorthosilicate (TMOS), SiO₂, Na₂SiO₃ or a combination thereof.

In a particular embodiment, the calcium is in the form of CaO, CaCO₃, Ca(OH)₂, Ca(NO₃)₂, calcium oxalate, calcium acetate, or a combination thereof.

In another embodiment, the magnesium is in the form of MgO, MgCO₃, Mg(OH)₂, Mg(NO₃)₂, magnesium oxalate, magnesium acetate, or a combination thereof.

In an embodiment, the Na₂O, Na₂CO₃, NaOH, NaNO₃, sodium oxalate, sodium acetate, or a combination thereof.

In a supplemental embodiment, the lithium is in the form of Li₂O, Li₂CO₃, LiOH, LiNO₃, lithium oxalate, lithium acetate, or a combination thereof.

In another embodiment, the potassium is in the form of K₂O, K₂CO₃, KOH, KNOB, potassium oxalate, potassium acetate, or a combination thereof.

In another embodiment, the glass comprises P₂O₅, H₃PO₄, NaH₂PO₄, Na₃PO₄, CaHPO₄, Ca₃(PO₄)₂, apatite, hydroxyapatite, or a combination thereof.

In an embodiment, further comprising an organic precursor.

In an embodiment, the organic precursor is triethyl phosphate (TEP).

In a further embodiment, the titanium is TiO₂.

In another embodiment, the powder mixture is melted by firstly heating at 870° C. for 20 min decomposing the Na₂CO₃, and subsequently to at least 1000° C. for 2.5 h.

In another embodiment, the powder mixture is melted by firstly heating at 870° C. for 20 min decomposing the Na₂CO₃, and subsequently to at least 1500° C. for 2.5 h.

In an embodiment, the melt is quenched by direct immersion in cold water or on a copper plate.

In a further embodiment, the mixture of Ti and silica comprises between 5 to 20 wt % of Ti.

In an embodiment, the glass comprises >5 mol % of TiO₂.

In an embodiment, the mixture of Ti and silica comprises at least 50 to 85 wt % of silica.

In another embodiment, the mixture of Ti and silica comprises at least 50 wt % of silica.

In a further embodiment, it is encompassed further comprising incorporating up to 10 wt % of phosphate (P) to the mixture to Ti and silica.

In a further embodiment, it is encompassed further comprising incorporating up to 28 wt % of sodium (Na) to the mixture to Ti and silica.

In a particular embodiment, the glass is a melt-derived bioactive glass or a sol-gel bioactive glass.

In an embodiment, the glass when immersed in a body fluid forms a titanium substituted hydroxyapatite hydroxyapatite (HA).

In an embodiment, wherein the glass provides enamel remineralization, enamel whitening and/or increases osteogenic differentiation.

In another embodiment, the enamel remineralization is for dentistry or bone tissue engineering.

### BRIEF DESCRIPTION OF THE DRAWINGS

Reference will now be made to the accompanying drawings.

FIG. 1 illustrates an overview of the sol-gel process for the synthesis of 70S in (A); 70S drying and calcination in (B); ground 70S in (C); 70STi synthesis in (D), 70STi gelation and calcination in (E); and ground 70STi in (F).

FIG. 2 illustrates the characterization of as-made glasses showing in (A) FTIR analysis; in (B) XRD diffractograms; in (C) XPS analysis of surface composition; and in (D) EDS surface mapping of elements in 70STi (*p<0.02).

FIG. 3 illustrates FTIR analysis of glasses immersed in SBF for AM, 1 day, 3 days, 7 days and 14 days for 70S in (A), and 70STi in (B).

FIG. 4A illustrates XPS analysis of surface composition (atomic %) as obtained through XPS survey spectra in 70S as made (AM) and after immersion in SBF for up to 14 days.

FIG. 4B illustrates XPS analysis of surface composition (atomic %) as obtained through XPS survey spectra in 70STi as made (AM) and after immersion in SBF for up to 14 days.

FIG. 4C illustrates elemental release from 70S and 70STi to SBF as obtained from ICP for Ti.

FIG. 4D illustrates elemental release from 70S and 70STi to SBF as obtained from ICP for Si.

FIG. 4E illustrates elemental release from 70S and 70STi to SBF as obtained from ICP for Ca.

FIG. 4F illustrates elemental release from 70S and 70STi to SBF as obtained from ICP for P.

FIG. 5 illustrates SEM morphological characterization of 70S and 70STi as a function of time in SBF, showing in (A) 70S AM, in (B) 70S 3 d immersion, in (C) 70S 7 d immersion, in (D) 70STi AM, in (E) 70STi 3 d immersion, and in (F) 70STi 7 d immersion.

FIG. 6 illustrates the elemental mapping of 70STi after 7 days of immersion in SBF.

FIG. 7 illustrates the experimental setup for remineralization of enamel samples with 70STi.

FIG. 8 illustrates the results of microhardness testing on enamel samples at the different stages of demineralization and remineralization.

FIG. 9 illustrates cell viability results for hDPSCs grown on tissue culture polystyrene as a control or in the presence of 70S and 70STi as measured for up to 5 days by an Alamar Blue assay.

FIG. 10 illustrates ALP activity of hDPSCs cultured in the presence of 70S and 70STi: control hDPSCs in non-osteogenic media (A), control hDPSCs in osteogenic media (B), hDPSCs in contact with 70S in osteogenic media (C), hDPSCs in contact with 70STi in osteogenic media (D).

FIG. 11 illustrates digital images of melt-derived glasses.

FIG. 12 illustrates XRD of melt-derived glass powders, wherein samples 1 and 2 are pure glasses, while samples 3, 4, and 5 show few crystalline domains corresponding to sodium, calcium silicates and/or Na₃PO₄.

FIG. 13 illustrates XRD of 60S10Ti, 65S7.5Ti and 60S10Ti—HNO₃.

FIG. 14 illustrates FT-IR of 60S10Ti and 65S7.5Ti.

FIG. 15 illustrates the atomic surface composition of 60S, 60S5Ti, 60S10Ti, 65S7.5Ti and 60S10Ti—HNO₃ measured by XPS.

FIG. 16 illustrates FT-IR spectra of 60S, 60S5Ti, 60S10Ti and 65S7.5Ti after testing in SBF for 1 hr, 6 hr, 1 d, 3 d and 7 d.

### DETAILED DESCRIPTION

In accordance with the present disclosure, there is provided a bioactive glass composition comprising titanium homogenously incorporated and distributed throughout the glass wherein the titanium is retained at the glass surface once the glass is in contact with a physiological solution.

The provided titanium-containing bioactive glass is aimed at generating titanium substituted hydroxyapatite. After 14 days immersion in simulated physiological fluid, the glass forms hydroxyapatite with titanium inclusions. Enamel remineralized with this glass is significantly harder than other enamels remineralized with bioactive glass in literature. The titanium-containing glass is non-toxic to cells and in fact promotes a response that is responsible for bone formation. From synthesis, to characterization of the mineralized layer, to cellular response, it is demonstrated that the encompassed composition of titanium-containing bioactive glass can be used for both enamel remineralization and application in the broader field of bone tissue engineering.

The enclosed formulation and composition have a slowed dissolution rate, to allow the titanium time to remain incorporated in the glass and then within the eventual hydroxyapatite layer. Titanium inclusion itself is non-obvious, with many potential issues arising during synthesis as titanium can act as a crystallization nucleator within the glass network. This has inhibited previous research in the area. The exemplified bioactive glass with titanium incorporation allows for retention of the titanium within the glass and eventual mineralized layer. This is in contrast to previous bioactive glass formulations with titanium inclusions that targeted titanium release to local physiological environment during the mineralization process.

Enamel is the hardest and most heavily mineralized material in the human body. It is 96% mineral (hydroxyapatite), 4% organic matter and a variety of trace metallic elements. The enamel layer is naturally susceptible to demineralization and, as it is not a living tissue, is unable to fully remineralize by biological methods. Demineralization causes issues in overall tooth health, ranging in severity from dental sensitivity to cavity formation. When placed in physiological fluid, bioactive glasses undergo a transformation from glass to hydroxyapatite, making bioactive glass a promising material to induce enamel remineralization. In fact, certain bioactive glasses are commercially available in over the counter toothpastes. However, these bioactive glasses simply replace the lost hydroxyapatite, without adding new properties to the remineralized layer. Doping the bioactive glass with metallic ions can impart desirable and unique properties that are not inherent to natural hydroxyapatite.

Titanium exists in trace amounts in native enamel and its presence has been correlated with increased tooth hardness and brightness. Synthetic titanium substituted hydroxyapatite exhibits better mechanical, antibacterial and aesthetic properties than unmodified hydroxyapatite. Due to these improved properties, and a significantly increased bone-forming cellular response, titanium-substituted hydroxyapatite has applications extending beyond dentistry and into the broader bone tissue engineering field.

It is provided the use of a sol-gel method to synthesize a titanium-containing silicate-based bioactive glass aimed at generating titanium substituted hydroxyapatite. Titanium is homogenously distributed throughout the glass, without modifying the structure or amorphous nature. After 14 days immersion in simulated body fluid, the glass forms a titanium substituted hydroxyapatite. It is disclosed that the titanium-containing glass is non-toxic to cells. Furthermore, cells in contact with titanium-containing bioactive glass exhibit significantly higher levels of differentiation towards bone-formation. Examination of enamel surfaces treated with the titanium-containing glass showed significantly increased microhardness compared to enamel surfaces treated with a control glass. From synthesis, to characterization of the mineralized layer, to cellular response our results demonstrate that this titanium-containing bioactive glass has potential both for enamel remineralization and application in the broader field of bone tissue engineering.

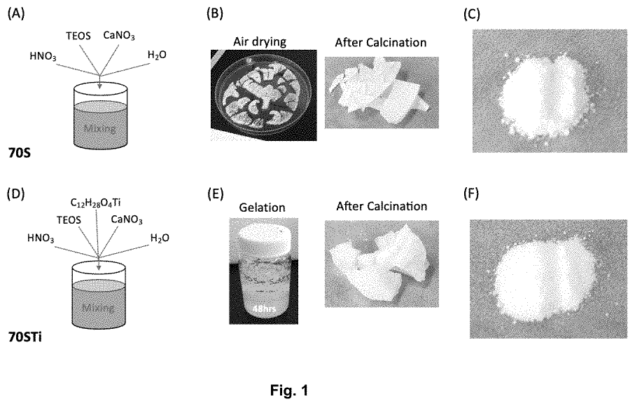

An overview of the sol-gel process as encompassed herewith to synthesize 70S and 70STi glass is illustrated in FIG. 1. Precursors are added to nitric acid and water, allowing for hydrolysis and polycondensation reactions to occur, forming 70S (FIG. 1A) and 70STi (FIG. 1D). 70S forms a very soft, pourable gel that dries clear (FIG. 1B). 70STi gel is incubated at 37° C. for 48 hrs, to allow for increased interaction between the free Ti and the glass network during gelation. The resulting gel is opalescent (see FIG. 1E). After calcination, the 70S glass (FIG. 1B) is brittle and easily breaks whereas the 70STi glass (FIG. 1E) retains more of its shape in the form of large clumps. Finally, 70S and 70STi were manually grind and sieve to obtain particles in the range of 25-45 μm. (FIGS. 1C and 1F respectively).

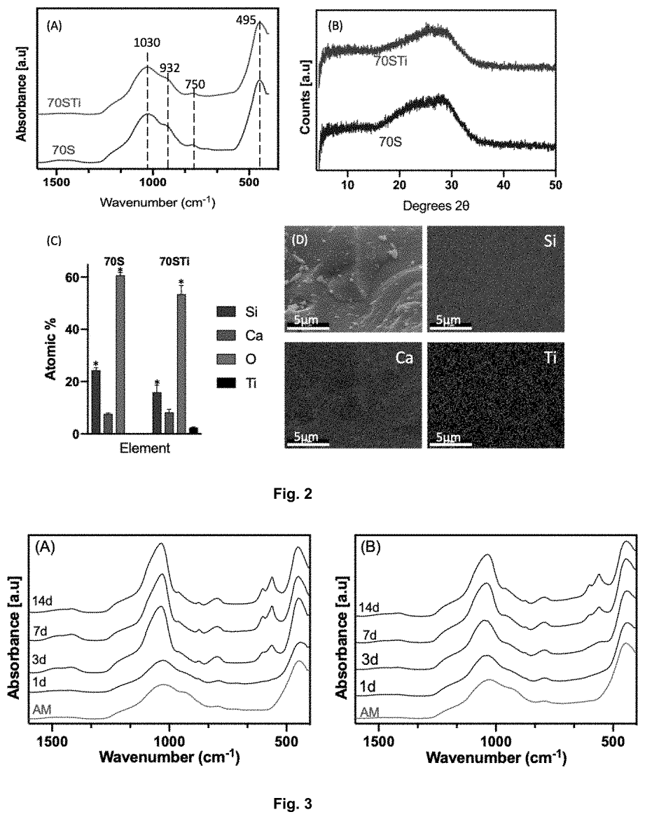

70S and 70STi were characterize to determine structure and composition. FIG. 2A shows the FTIR spectra of the control 70S and modified 70STi glasses. Both glasses exhibit peaks characteristic of silicate-based glasses. Silicate groups can give peaks attributed to either bridging oxygen (BO) or non-bridging oxygen (NBO) groups. The peaks at 495 cm⁻¹, 750 cm⁻¹, 932 cm⁻¹ and 1030 cm⁻¹ can be assigned respectively to δ_{(Si—O—Si)}, v_{s(Si—O—Si)} of BO groups between tetrahedrons, v_{as(Si—O—Si)} of NBO groups and v_{s(Si—O—Si)} of BOs within the tetrahedrons. The spectra of 70S is consistent with those reported for glasses of the same composition and calcination temperature (Roman et al., 2003, Chemistry of Materials, 15: 798-806). In 70STi there is no additional peak that can be attributed to the presence of a Ti species present in the glass structure. This mirrors previous work on the insertion of Ti ions into synthetic HA and bioactive glass, where the inclusion of Ti is not directly observable in FTIR results.

XRD was performed to determine if the introduction of Ti induces crystallinity in the material. While Ti can act as a nucleation point for crystallization within glass, the exemplified 70STi remains amorphous even after high temperature calcination, as shown in FIG. 2B. Both glasses exhibit the amorphous “halo” effect centered around 29° 2θ, characteristic of amorphous or highly disordered structures. This contrasts to previous bioactive glasses with Ti inclusion, where crystallinity was nucleated through Ti introduction.

Using FTIR and XRD, is was not possible to directly observe Ti inclusion in the glass. However, Ti was seen on the surface of all 70STi samples through XPS (FIG. 2C), which shows 2.32±0.01 at % Ti. The Si at % and O at % are significantly (p<0.005) higher for 70S compared to 70STi. The difference in Ca in % is not significant between the two compositions. EDS mapping (FIG. 2D) confirms the homogenous distribution of elements throughout the material. Si, Ca and Ti are evenly distributed throughout the sample, which indicates that Ti is incorporated into the glass structure homogeneously and not agglomerated at certain points.

Particle size and specific surface area are important factors as they impact on the reaction kinetics once the powders are immersed in body fluids. The median particle size was consistent across both glasses, as shown in Table 1. The size distributions were also consistent.

TABLE 1
Glass particle textural properties

| | Particle Size | SSA | Average Pore | Average Pore |
|---|---|---|---|---|
| Sample ID | (μm) | (m²/g) | Width (nm) | Volume (cm³/g) |
| 70S | 36 ± 4 | 69 ± 4 * | 9.0 ± 0.1** | 0.17 ± 0.01 |
| 70STi | 36 ± 2 | 58 ± 5 * | 10.1 ± 0.1** | 0.16 ± 0.01 |

(n = 3): average particle diameter, specific surface area (SSA), average pore width, and average pore volume,
* p < 0.02,
** p < 0.0001

Both glasses show high specific surface area (SSA) values, comparable to previous sol-gel derived bioactive glasses. However, there is a significant difference in SSA between 70S and 70STi. 70S has a higher SSA (69±4 m²/g) than the modified glass (58±5 m²/g) (Table 1). This decrease in surface area could be attributed to the inclusion of Ti in the glass composition or to the extended gelation period of the modified glass. Extended gelation has been previously shown to reduce SSA.

The bioactivity of 70S and 70STi were investigated for up to 14 days in SBF (FIG. 3). In both glasses, there is an observable shift in the 1035 cm⁻¹ peak relative to v_{s(Si—O—Si)} of BO groups between silica tetrahedrons towards lower wavenumbers; this is indicative of the formation of phosphate bonds, with v(PO43-) centered at ˜1020 cm⁻¹. In 70S, peaks at 570 cm⁻¹ and 600 cm⁻¹ are observed after 3 days of mineralization. These are assigned to the bending modes of v4(P—O—P) from the PO₄³⁻ group. For 70STi, this first presents as a broad band around 600 cm-1 after 3 days of mineralization. This band then separates after 7 days of mineralization to form the two distinct peaks seen in 70S.

The band at 800 cm⁻¹, associated with vs(Si—O—Si), increases throughout immersion in SBF on both glasses, indicative of the formation of a silica gel layer on their surface.

After one day of immersion for 70S and 3 days for 70STi a peak at 870 cm⁻¹ appears. This is assigned to v2(CO₃²⁻), associated with carbonated apatite. The broad bands at 1470 and 1425 cm⁻¹ are attributed to the stretching modes, v1 and v3 respectively, of CO₃²⁻.

Spectra for both glasses at 14 days of immersion are overall similar and exhibit equivalent peaks, however 70S presented more defined characteristic carbonate and phosphate indicators at earlier time points. Several factors may delay the formation of HA on the modified glass at earlier time points: as shown in Table 1, 70STi has lower surface area than 70S. The reduced surface area of 70STi may result in slower HA formation compared to 70S. Additionally, previous work indicates that the presence of Ti within the glass network alters its dissolution rate. Ti is a highly charged tetravalent cation, allowing it to act as both a network former and network modifier in the glass. When acting as a network former, Ti will create additional BOs due to being the same valence as Si, which reduce glass solubility. When substituting Ti⁴⁺ ions for Ca²⁺ ions bonds, Ti—O bonds are formed with a more covalent character than Ca—O bonds.

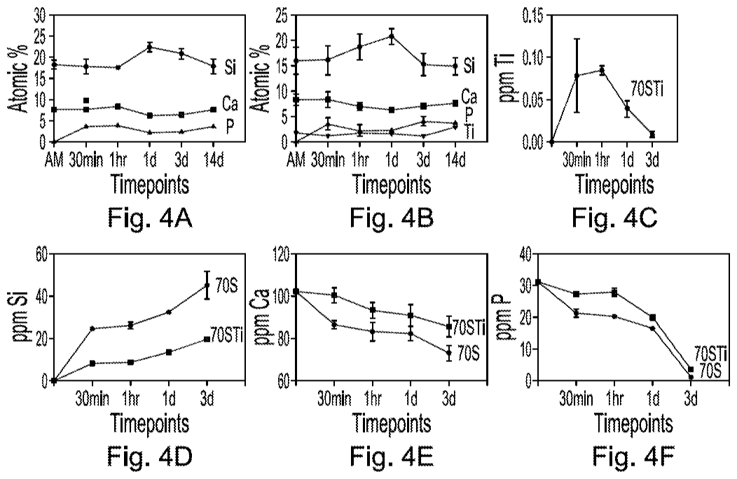

XPS analysis of the surface of the glass powders (FIGS. 4A and B) confirms the formation of a calcium-phosphate rich layer after immersion in SBF. A strong indication for this is the presence of P on the glass surface after immersion. For both 70S and 70STi, no P was observes on the surface of the as made (AM) glass, as it is not included in the composition. However, P appears after only 30 minutes of immersion for both samples while Ca remains constant, indicating the initial stages of HA formation on both glass surfaces. Si at % on 70S does not significantly change from the AM value (18±1 at %) until one day of immersion when it increases to 22±1 at % (FIG. 4A). By 14 days of immersion, Si at % has fallen significantly to 18±2 at %. A similar trend was seen for 70STi (FIG. 4B). There is no significant difference in Si at % until 1 day of immersion, going from 15±3 at % for the AM glass to 21±2 at % after 1 day of immersion. Between 1 and 3 days of immersion, there is a significant decrease to 15±2 at %, with no significant change up to 14 days of immersion. The trend of increase and then decrease in Si content observed for both glasses is consistent with previous works that show the formation of a silica rich layer during the first stage of the reaction which is then covered by a HA layer at later timepoints.

Ti is observable on the surface of 70STi at every time point (FIG. 4B), implying that it is present in the AM glass and in the eventual HA that begins to form after 3 days of immersion. There is no significant difference in Ti at % between the AM glass and the glass after 3 days of immersion. However, between 3 and 14 days of immersion, there is a significant increase in the amount of Ti on the glass surface, which seems to indicate accumulation of Ti on the HA layer formed on 70STi.

Ti release to SBF is shown in FIG. 4C. At time zero, Ti in solution is not detected as Ti is not inherent to SBF. After 30 minutes of immersion, 0.07±0.04 ppm of Ti are released into solution. By 3 days of immersion, the levels of Ti released significantly decreases as the level approaches zero.

Ti release at all time points is very low, indicating that most of the Ti is retained within the glass initially and then within the HA layer at later time points. Previous bioactive glass compositions that included metallic ions saw an increased release of metallic ions to solution over time. More specifically, studies on glasses with similar amounts of Ti inclusion show prolonged and increasing release of Ti (around 4 ppm) up to 30 days immersion in SBF, in contrast to the decreasing trend seen for the composition described herein. The disclosed glass retains Ti for two reasons. The high silica content of the glass slows the transformation to HA, allowing more time for Ti to integrate in the forming HA rather than release to solutions. Additionally, the sol-gel processing described includes a 48 hr gelation step. This extended gelation is absent from other sol-gel based Ti bioactive glasses. This allows Ti to have greater interaction with the glass network before drying and calcination, improving Ti retention in the glass and eventual HA layer.

Beyond looking at Ti release to solution, ICP analysis is a valuable secondary method to confirm the formation of HA on the glass. In FIG. 4D the release of Si is seen from the glass to SBF across all time points, with increasing Si concentration in solution over time. 70S consistently releases more Si over three days of immersion compared to 70STi. The release of Si to solution is an indicator of glass network dissolution and has previously shown to be a reliable indicator of bioactivity. A decrease in Ca and P in solution is a good indicator of bioactivity in glass. Ca release for 70S and 70STi is similar across all time points (FIG. 4E). For 70S, there is a significant decrease in Ca release after only 30 minutes. The decrease from the baseline Ca concentration for 70STi is not significant until 1 hr of immersion. For both compositions, there is no significant change between 1 hour and 1 day of immersion. 70S shows a further significant decrease from 1 day to 3 days. This period does not see a significant decrease for 70STi. 70S draws a significantly higher amount of Ca from solution compared to 70STi. As P is not inherent to the glass compositions, no P will come from the glass itself, instead being drawn from SBF to the surface of the glass to combine with Ca to form HA. A decrease in P ppm in solution was observed for both glass compositions over time (FIG. 4F). By 1 day of immersion, the difference in P levels in solution between 70S and 70STi is no longer significant. By 3 days of immersion, the concentration of P in solution for both 70S and 70STi converge to approximately 2 ppm. These results as a whole are consistent with the observed results from XPS and FTIR that indicate while that both glasses form HA, 70S does this at a faster rate, due to increased bioactivity compared to 70STi.

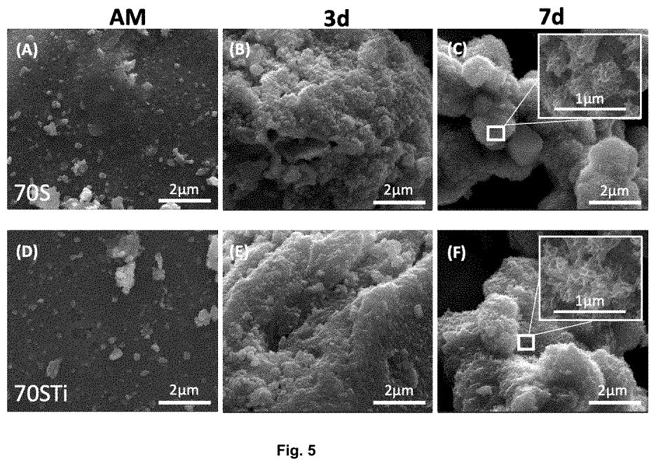

The formation of HA is also confirmed by SEM (FIG. 5). The AM glasses are relatively smooth, with smaller particles adhered to the surface of the larger particles (FIGS. 5A and D). There is no significant difference in appearance between 70S and 70STi. After 3 days in SBF, the surfaces of the glasses are considerably coarser and the smaller particles that are present for the AM samples are no longer present, likely having been washed off by immersion in SBF. The surface of 70S (FIG. 5B) appears more roughened than 70STi (FIG. 5E), echoing previous observations from FTIR, XPS, and ICP that 70STi is slower to develop HA.

By 7 days in SBF (FIG. 5C and F), the surfaces of both glasses are fully covered by developed flakey crystals, arranged in larger globule-type structures. This morphology is an indicator of HA formation. There is no significant difference between 70S and 70STi by 7 days. This contrasts to previous work on Ti-containing bioactive glass where the morphology of the developed HA was significantly different to the HA on the surface of unmodified glass, showing this difference after only 3 days of immersion in 1×SBF: HA crystals on the Ti-containing 58S glass were arranged in worm-like patterns between 3 and 5 days of immersion rather than the globule morphology seen on 58S unmodified glass.

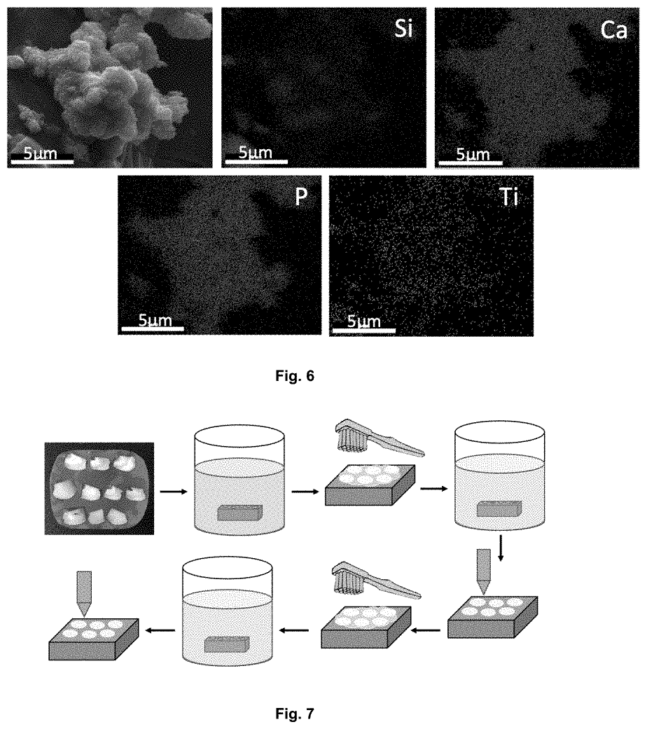

FIG. 6 shows the elemental mapping of 70STi after 7 days of immersion in SBF. Ca and P are distributed evenly across the material. The uptake of P indicates the formation of the calcium phosphate layer. Ti mapping indicates that Ti remains present during immersion in SBF and remains evenly distributed throughout the material. These results suggest the formation of Ti containing HA layer on the glass surface, confirming previous characterization.

Having demonstrated the ability of 70STi to mineralize to form a Ti-HA, the application of using 70STi to remineralize dental enamel was examined. This is undertaken using the experimental setup shown in FIG. 7 where enamel samples are subjected to successive demineralization and remineralization cycles, with hardness measured at each point.

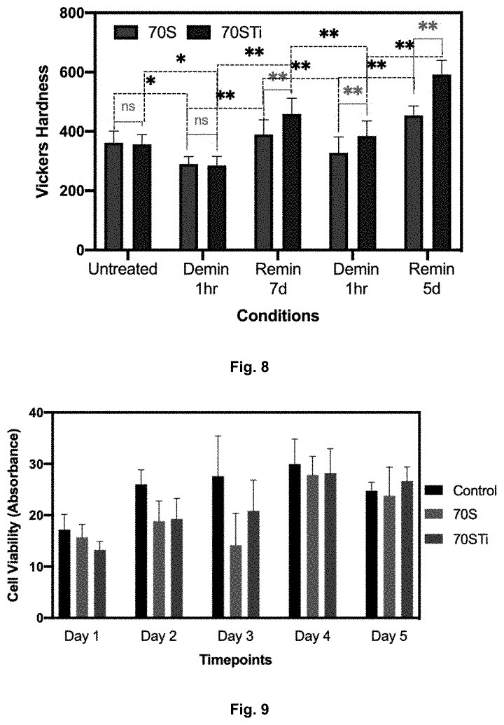

In FIG. 8, the results of microhardness testing are seen on enamel samples at the different stages of demineralization and remineralization. It is seen that one hour of demineralization in orange juice is sufficient to significantly reduce the hardness of enamel in both pucks. After brushing with 70S and 70STi and a remineralization period of 7 days, there is a significant increase in hardness for both 70S and 70STi treated enamel relative to the demineralized values. Enamel samples treated with 70STi are significantly harder than samples treated with 70S after this remineralization period. Subjecting both pucks to a 1 hr acid challenge results in significant reductions in hardness for both groups. However, 70STi treated enamel remains significantly harder than 70S treated enamel and neither group demineralizes to original demineralization values. A further treatment with bioactive glass slurry and remineralization for 5 days results, once again, in a significant increase in hardness for both groups. 70STi treated enamel is again significantly harder than 70S treated enamel. The final remineralized hardness for 70STi treated enamel is a value (592±48) that is notably higher than hardness values reported in literature for similar studies examining enamel remineralization with bioactive glass. These results demonstrate the effectiveness of 70STi to remineralize enamel while improve surface properties beyond what has been previously explore for bioactive glass remineralization.

It is critical to the potential commercial applications of 70STi that it be biocompatible. As such, cellular studies were conducted to look at viability of cells in contact with the glass and mineralization experiments to assess how 70STi influences osteoblast proliferation. FIG. 9 shows cell viability results for AM 70S (1 mg/well) and 70STi (1 mg/well) in contact with hDPSCs for time points up to 5 days. The control samples are hDPSCs grown on tissue culture polystyrene. Between day 1 and day 5 there is a statistically significant increase (p<0.04) in absorbance in the control, 70S and 70STi wells indicating continued and sustained cell viability as the hDPSCs proliferate. In ICP results, it was observed that 70STi releases small amounts of Ti to solution at early mineralization time points. However, from FIG. 9 it is observed that there is no significant difference in cell viability between the control hDPSCs and the hDPSCs in contact with 70S and 70STi at any time point. This result indicates that the presence of Ti in solution at early time points does not have a negative effect on cell viability.

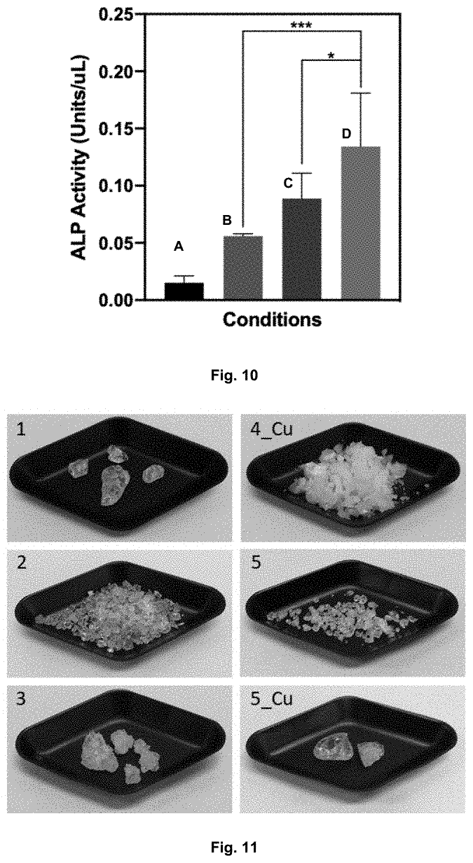

Alkaline phosphatase activity (ALP) is an early cell marker for osteoblasts, as it initiates the process of osteoid calcification. Increasing ALP activity in cell assays marks the transition toward osteoblastic differentiation. FIG. 10 shows the results for ALP activity in hDPSCs, cultured in the presence of 70S and 70STi. hDPSCS grown in non-osteogenic media (A), give low levels of ALP activity. With the addition of osteogenic factors to the media (B), hDPSCs show significantly more ALP activity (p<0.03). hDPSCs in osteogenic media in the presence of 70S (C) and 70STi (D) show higher levels of ALP activity. There is a significant (p<0.002) increase in ALP activity for hDPSCs in contact with 70STi in osteogenic media compared to control cells in osteogenic media. There is also a significant (p<0.032) difference between hDPSCs in contact with 70S and 70STi, indicating that the presence of Ti on the material surface is the key factor in the increased ALP activity.

An increase in ALP activity in the presence of bioactive glass is well established, as the ionic dissolution products released during HA formation promote osteogenic differentiation. In addition, previous works examining the inclusion of Ti in synthetic HA show that the presence of Ti on the material surface induces significantly more osteogenic differentiation compared to unmodified HA. The provided results show that this increase in osteogenic differentiation ability holds true for 70STi as it undergoes the transformation to a Ti-substituted HA layer.

It is also disclosed melt-derived bioactive glasses that contain Titanium which are bioactive and yet are able to retain Ti at its surface in the form of a Ti-substituted HA. As encompassed are melt-derived preparations which are easier to standardize and therefore to commercialize than sol-gel preparations.

The non-obvious aspect of the preparation of a melt-derived bioactive glass was how to design a composition that would be still bioactive (Ti is known to slow down bioactivity) and yet not release all the Ti in solution. To make a melt-derived glass bioactive one needs to add enough Na or Ca to disrupt the glass network. This may risk having Ti ions also be lost in solution.

Accordingly, a few compositions are disclosed that go all to way from very low Na/Ca (25 mol % CaO+Na₂O) to low Na/Ca (35 mol % CaO+Na₂O) and tested to measure their bioactivity and formation of Ti-HA on their surface by XPS.

The glass compositions reported in Table 2 are prepared using a traditional melt-quenching technique. To obtain these compositions, high purity precursors SiO₂, Na₂CO₃, CaO, P₂O₅ and TiO₂ powders (>99.5%, Sigma-Aldrich) are mixed and ground in a mortar for homogenization, then placed in a Pt crucible and heated between 1000 and 1500° C. in a muffle furnace. T ramp: 15° C./min, 1^{st} plateau at 870° C. for 20 min to decompose Na₂CO₃, 2^{nd} plateau at 1500° C. for 2.5 h (1000° C., in the case of samples 4 and 5). The melts are quenched by direct immersion in cold water or/and on a copper plate. Samples named just with a number are quenched in water, the ones labelled with a number followed by ‘Cu’ were quenched on copper. The resulting glasses, shown in FIG. 11, are then crushed and reduced to powder with particle sizes between 25 and 45 microns. Before grinding, samples 1, 2, and 5 are transparent with yellow to gray shades. Samples 3 and 4 Cu are translucent white. After grinding, all the samples are white powders.

TABLE 2
Composition of the melt-quenched glasses synthesized.

| Glass | mol % composition | | | | |
|---|---|---|---|---|---|
| sample | SiO₂ | Na₂O* | CaO | P₂O₅ | TiO₂ |
| 1 | 70 | 10 | 15 | 0 | 5 |
| 2 | 65 | 10 | 15 | 0 | 10 |
| 3 | 60 | 25 | 7 | 2 | 6 |
| 4 | 60 | 28 | 0 | 5 | 7 |
| 5 | 50 | 15 | 20 | 5 | 10 |

*The precursor Na₂CO₃ is converted to an equivalent molar amount of Na₂O during the melting process.

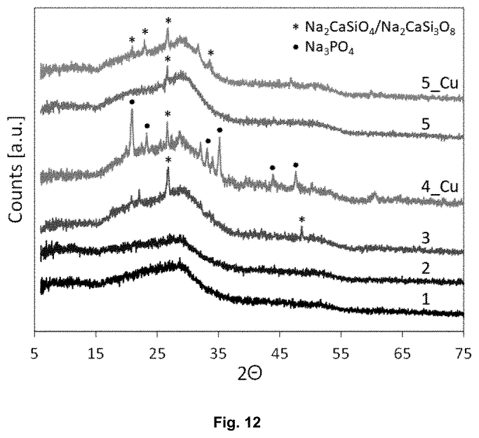

X-Ray diffractometry (FIG. 12) shows that just samples 1 and 2 are pure glasses, while samples 3, 4, and 5 show few crystalline domains corresponding to sodium, calcium silicates and/or Na₃PO₄ (in particular sample 4). The quenching in water hinders the formation of crystalline phases, because of the faster drop in temperature. However, in samples 3 and 5 the water quenching is not able to completely impede formation of crystalline domains.

The in vitro bioactivity of the samples are investigated by analysis of apatite/hydroxyapatite formation ability in a simulated body fluid (SBF) solution. The powders are immersed in 1×SBF for 1 week and their surface change are investigated after 3 and 7 days, through XPS analysis (samples labelled with the number followed by _3 d or _7 d, respectively). Table 3 reports the elemental surface composition of the samples, quantified by XPS. In parenthesis the theoretical/expected compositional values are reported. The results show that all the samples exhibit an increase of surface P and Ca after immersion in SBF, which could be related with the formation of calcium phosphate minerals. As expected, the most “bioactive” samples are those with a higher content of Na (i.e. 3 and 4), which are rapidly released in the SBF solution. Neither the presence of Ca and P in the original glass composition appears critical for the accumulation of a surface layer richer in Ca and P. As seen on the sol-gel glasses, Titanium tends to accumulate on the surface after immersion in SBF, most likely because of the crosslinking nature of the tetravalent Ti⁴⁺ ion in the glass network. We compared our samples with a benchmark bioactive glass, 45S5 (Bioglass®) doped with 5% mol Titanium (45S5-Ti, reported in the last 3 rows in Table 3). The results show that Ti-doped Bioglass® is remarkably more active in SBF compared to all our samples. However its rapid dissolution in SBF hinders the accumulation of Titanium on the surface, which always appears 2 to 3 times less concentrated than in all our glasses.

TABLE 3
Elemental surface composition of the melt-quenched glasses quantified by XPS.

| | % elemental composition | | | | | | |
|---|---|---|---|---|---|---|---|
| Sample | O | Si | Ca | Na | Ti | P | Mg |
| 1 | 63.2 ± 0.1 | 23.5 ± 0.2 | 3.8 ± 0.0 | 7.8 ± 0.3 | 1.6 ± 0.1 | 0.0 ± 0.0 | 0.0 ± 0.0 |
| | (60.8) | (23.3) | (7.5) | (6.7) | (1.7) | (0.0) | (0.0) |
| 1_3d | 64.4 ± 0.3 | 14.5 ± 0.3 | 6.2 ± 0.2 | 1.9 ± 0.1 | 5.7 ± 0.1 | 6.6 ± 0.4 | 0.7 ± 0.3 |
| 1_7d | 66.6 ± 0.1 | 8.0 ± 0.6 | 6.6 ± 0.1 | 1.3 ± 0.3 | 8.5 ± 0.3 | 8.3 ± 0.0 | 0.8 ± 0.2 |
| 2 | 63.1 ± 0.3 | 22.1 ± 0.1 | 4.0 ± 0.0 | 7.8 ± 0.1 | 2.9 ± 0.1 | 0.0 ± 0.0 | 0.0 ± 0.0 |
| | (60.8) | (21.7) | (7.5) | (6.7) | (3.3) | (0.0) | (0.0) |
| 2_3d | 63.3 ± 0.4 | 16.1 ± 0.5 | 6.3 ± 0.1 | 1.6 ± 0.2 | 5.3 ± 0.1 | 6.2 ± 0.4 | 1.2 ± 0.6 |
| 2_7d | 66.0 ± 0.4 | 9.0 ± 0.4 | 7.0 ± 0.2 | 1.2 ± 0.1 | 7.9 ± 0.4 | 8.2 ± 0.2 | 0.6 ± 0.1 |
| 3 | 60.1 ± 0.1 | 18.5 ± 0.4 | 1.9 ± 0.1 | 17.4 ± 0.2 | 1.2 ± 0.2 | 0.9 ± 0.1 | 0.0 ± 0.0 |
| | (57.3) | (20.0) | (3.5) | (16.7) | (2.0) | (0.6) | (0.0) |
| 3_3d | 64.7 ± 0.3 | 7.9 ± 0.2 | 9.7 ± 0.2 | 1.2 ± 0.1 | 5.4 ± 0.2 | 9.8 ± 0.4 | 1.5 ± 0.1 |
| 3_7d | 65.4 ± 0.3 | 5.2 ± 0.6 | 9.5 ± 0.3 | 1.1 ± 0.1 | 6.8 ± 0.2 | 10.6 ± 0.2 | 1.4 ± 0.2 |
| 4_Cu | 62.2 ± 0.1 | 11.9 ± 0.1 | 0.0 ± 0.0 | 20.6 ± 0.3 | 1.3 ± 0.1 | 4.0 ± 0.1 | 0.0 ± 0.0 |
| | (57.6) | (20.0) | (0.0) | (18.7) | (2.3) | (1.4) | (0.0) |
| 4_Cu_3d | 64.1 ± 0.4 | 2.0 ± 0.1 | 13.0 ± 0.1 | 0.9 ± 0.2 | 3.6 ± 0.2 | 14.1 ± 0.3 | 2.3 ± 0.1 |
| 4_Cu_7d | 64.7 ± 0.1 | 1.7 ± 0.1 | 12.3 ± 0.1 | 0.9 ± 0.1 | 4.8 ± 0.2 | 13.8 ± 0.3 | 1.9 ± 0.3 |
| 5 | 62.1 ± 0.2 | 15.2 ± 0.1 | 4.5 ± 0.2 | 12.9 ± 0.2 | 2.4 ± 0.1 | 2.9 ± 0.2 | 0.0 ± 0.0 |
| | (58.6) | (16.7) | (10.0) | (10.0) | (3.3) | (1.4) | (0.0) |
| 5_Cu | 61.6 ± 0.5 | 16.2 ± 0.2 | 4.7 ± 0.0 | 11.6 ± 0.2 | 2.7 ± 0.2 | 3.2 ± 0.1 | 0.0 ± 0.0 |
| | (58.6) | (16.7) | (10.0) | (10.0) | (3.3) | (1.4) | (0.0) |
| 5_3d | 66.5 ± 0.3 | 7.9 ± 0.1 | 7.5 ± 0.1 | 2.2 ± 0.4 | 7.1 ± 0.1 | 8.6 ± 0.2 | 0.3 ± 0.1 |
| 45S5-Ti | 60.7 ± 0.2 | 11.9 ± 0.2 | 4.8 ± 0.1 | 17.0 ± 0.0 | 1.3 ± 0.3 | 4.3 ± 0.2 | 0.0 ± 0.0 |
| | (55.5) | (14.3) | (11.5) | (15.3) | (1.7) | (1.7) | (0.0) |
| 45S5-Ti_3d | 63.8 ± 0.3 | 2.7 ± 0.1 | 15.3 ± 0.1 | 0.6 ± 0.5 | 2.0 ± 0.1 | 13.9 ± 0.3 | 1.7 ± 0.2 |
| 45S5-Ti_7d | 64.4 ± 0.1 | 2.1 ± 0.1 | 14.6 ± 0.1 | 0.1 ± 0.2 | 3.1 ± 0.2 | 14.0 ± 0.1 | 1.6 ± 0.2 |

* Some bioactivity tests involving this sample are still ongoing.

The rate of HA layer formation is highly dependent on glass degradation. Accordingly, the presence of Na₂O and other alkali or alkaline earth metals increases the rate of HA layer formation. High silica content and the presence of Ti reduce the rate of glass breakdown and hence the apatite layer formation rate. Due to their texture (pore size/volume and high surface area), sol-gel-produced glasses show higher bioactivity than the melt-quenched counterparts. In fact, the silica content can reach 85 mol % in sol-gel without reducing the bioactivity, while a maximum value of 70 mol % is allowed in melt-quenched glasses.

As exemplified herein, the sol-gel glass comprises 3.4 mol % TiO₂. Larger of TiO₂ tend to induce crystallinity in the final glass. One issue for why larger fraction of TiO₂ induce crystallinity of the final product is that an acid is used to catalyze the sol-gel synthesis (for example HNO₃). It is provided herewith that a basic catalyst (e.g. NaOH, NH₄OH or CaOH₂) allows producing glasses with larger final TiO₂% in their composition. It is thus provided a mean to synthesize bioactive sol-gel glasses with large TiO₂%, including Ca and if desired P in their compositions, as a basic catalyst, and the resulting glasses have better bioactivity than previously reported acid-catalyzed Ti-containing glasses.

It is thus disclosed and provided a Ti containing silicate-based bioactive glass, 70STi, with excellent in vitro mineralization and osteogenesis performance through the sol-gel method. Very little Ti is lost to solution after immersion in SBF, and the presence of Ti is observed on the surface of the material at all time points during the formation of the HA layer. This indicates that the Ti is retained within the glass and results in the production of a Ti substituted HA layer. Furthermore, this material is showed ability to remineralize enamel and impart high surface hardness. 70STi is not only fully cytocompatible, but also significantly increases osteogenic differentiation when compared to 70S.

It is further provided melt-derived Ti-containing bioactive glasses. As demonstrated, all the melt-derived bioactive glasses tested, included the ones that had some crystalline phases in them, were able to form a Ca/P rich layer containing Ti on their surface after just 3 days of SBF immersion. Accordingly, it is provided a mean to create melt-derived bioactive glasses that contain Ti, which are bioactive and yet allow for the creation of a Ti-rich HA layer on the surface.

It is encompassed that 70STi and melt-derived Ti-containing glasses proposed above have applications that span both in dentistry and the broader field of bone tissue engineering. It could act as an active repair agent for hard tissue, as it releases ionic dissolution products that promote cellular and genetic responses favorable to mineralization similarly to Bioglass®, while the presence of Ti on the surface of the glass and of the HA layer that gets deposited on it upon mineralization promotes cell adhesion and differentiation.

In an embodiment, the sol-gel silicate as described herein are synthesized based glass containing 10 mol % TiO₂ that is completely amorphous, with titanium existing as part of the network and not clusters of TiO₂, and able to form crystalline hydroxyapatite by day 3. This was never reported in the literature, and was made possible by the use of Ca(OH)₂ as a basic catalyst during the synthesis.

It is also encompassed that the sol-gel 70STi composition as described herein has a whitening effect on enamel. This was observed visually when 70STi glass was included in a water-based paste mimicking toothpaste and used in demineralization/remineralization experiments such as those shown in FIG. 7. Accordingly, it is encompassed a toothpaste composition comprising the Ti-containing bioactive glass as described herein.

### EXAMPLE I

### Glasses Fabrication and Characterization

Nitric acid (>99%), calcium nitrate tetrahydrate (>99%), tetraethyl orthosilicate (TEOS) (>99%) and titanium isopropoxide (>99%) are purchased from Sigma Aldrich (Canada).

### Sol-Gel Processing

A 70S control glass (71.4 wt % SiO₂-28.6 wt % CaO) and 70STi modified glass were fabricated (71.4 wt % SiO₂-23.6 wt % CaO-5 wt % TiO₂).

Nitric acid (12M) was added to deionised water at room temperature in a Teflon beaker and magnetically stir for 5 minutes. Over a period of 5 minutes, TEOS was added dropwise, and the solution magnetically stirred for a further hour at room temperature. At this point, calcium nitrate powder was added slowly, allowing it to dissolve before the following addition and stirred for a further hour. For the control glass 70S, the resulting sol was cast into polystyrene petri dishes and air dry at room temperature for 24 hours.

For the modified glass, titanium isopropoxide was added dropwise following the addition of calcium nitrate and subsequent stirring period. After the final addition, the solution was mixed for a further 30 minutes or until the viscosity is too high for any further stirring. The sol is cast into polypropylene vials, sealed and stored at 37° C. for further gelation and aging for 48 hours. The gel is removed and placed in open petri dishes to air dry at room temperature for further 24 hours.

Both the control and modified air-dried gels were calcined at 700° C. The temperature is reached at a heating rate of 3° C./min, followed by a 6-hour dwell time and cooled to room temperature. Following calcination, the glass was manually grinded and sieved to isolate a particle size range of 25-45 μm. The glass particles were stored in a desiccator until analysis.

### Particle Characterization

The particle size (D50) of the sieved glass powders was determined using a Horiba LA-920 (ATS Scientific Ink., Canada). The powders are dispersed in isopropanol before introduction into the machine. Three runs per powder sample were performed.

The specific surface areas of the powders are measured with nitrogen gas adsorption and desorption isotherms collected using a Micrometrics TriStar II Plus 2.03 gas sorption system. Isotherms are analyzed using the Brunauer-Emmett-Teller (BET) method to understand surface area. Pore size is determined using the Barrett, Joyner and Halenda (BJH) method (Mukundan et al., 2013, Biomatter 3(2): e24288). Prior to surface area measurements, the samples are degassed for 12 hrs at 150° C. under vacuum. Three runs are performed per powder sample.

### X-Ray Diffraction

X-ray diffraction (XRD) diffractograms of the glasses are analyzed with a Bruker D8 Discover X-ray diffractometer (Bruker AXSS Inc., USA) with a CuKα (λ=0.15406 nm) with power levels set to 40 mV and 40 mA. Three frames of 25° each are collected from 15-75° 2θ with an area detector and merged in post processing.

### Fourier Transform Infrared Spectroscopy

Fourier transform infrared spectroscopy (FTIR) is carried out on a Bruker Alpha II spectrometer, in ATR-FTIR mode, equipped with a deuterated-triglycine sulfate (DTGS) detector. The FTIR spectrometer resolution is set to 4cm⁻¹, the scan number to 128, and the scanner velocity is set to 10.0 kHz. All samples were analyzed in powder form, placed directly on the spectrometer.

### X-Ray Photoelectron Spectroscopy

X-ray photoelectron spectroscopy (XPS) is carried out using a Thermo Scientific Kα spectrometer, equipped with an Al cathode with incident Kα radiation of 1486 eV and a spot diameter of 200 μm. A low energy electron flood gun is used to prevent surface charging. Three points are selected for analysis for each sample. Data processing is performed using Avantage Software.

### Inductively Coupled Plasma Optical Emission Spectrometry

Release of Si, Ca, P, and Ti ions from the glass powders in simulated body fluid at several timepoints is measured using an inductively coupled plasma-optical emission spectrophotometer (ICP-OES, Thermo Scientific iCAP 6500, USA). Aliquots of 10 mL are filtered through a 0.2 μm nylon sieve and stored in 15 mL falcon tubes. To each of these vials, 5 ml of 45 w/v % nitric acid is added and incubated at 60° C. for 2 hours. Three samples for each timepoint and powder were analysed.

### Scanning Electron Microscopy

All samples are sputter coated (Edwards Auto 306 Cryo Evaporator) with a carbon coating (20 nm thickness). Scanning electron microscopy (SEM) images of the surface of the materials are taken with a field emission SEM (FEI Inspect F-50) under an accelerating voltage of 5 kV. Each sample is imaged in at least three different areas.

### Bioactivity

The in vitro mineralization abilities of the control and modified glasses is examined with Kokubo's simulated body fluid (SBF). Glass is added to 50mL falcon tubes at a 1.5 mg/mL glass to SBF ratio. Once a day, the vials are gently shaken to avoid agglomeration of the glass powder. Timepoints of 30 min, 1 h, 6 h, 1 d, 3 d, 7 d and 14 d are examined. Upon completion of the time point, the powders are rinsed with deionised water and then with 70% ethanol and dried overnight in an open falcon tube at 37° C.

### Cells

The human dental pulp stem cells (HDPSCs) are collected from permanent teeth and used at passage 3 to 6, as previously described (Bakkar et al., 2017, “Simplified and Systematic Method to Isolate, Culture, and Characterize Multiple Types of Human Dental Stem Cells from a Single Tooth, in: P. Di Nardo, S. Dhingra, D. K. Singla (Eds.)”, Adult Stem Cells: Methods and Protocols, Springer New York, New York, N.Y., pp. 191-207). The cells are cultured in α-MEM supplemented with 10% (v/v) fetal bovine serum (FBS) and 1% (v/v) Penicillin/Streptomycin. They are incubated in a vented petri dish at 37° C., 5% CO₂ until 90% confluence. Cells are then trypsinized, counted and seeded into 96 well plates (3000 cells/well).

### Bioactive Glass Powder Preparation for Cell Experiments

For all experiments, the glasses are used as made. Before contact with cells, the glasses are sterilized by immersion in 100% anhydrous ethanol for 20 minutes. Following this, the excess ethanol is removed, and the glass is left to dry overnight in a biological safety cabinet.

### Cell Viability

Human mesenchymal stem cells (1500/well) are put in contact with 1 mg bioactive glass/well. At designated time points (1 d, 2 d, 3 d, 4 d, and 5 d), the cell media is aspirated from the well plates. 150 μL of α-MEM supplemented with 10% (v/v) fetal bovine serum (FBS) and 1% (v/v) Penicillin/Streptomycin with a further addition of 10% (v/v) Alamar Blue is added to each well. 3 wells for each time point are used without cells as a blank. Following incubation of the well plate 37° C. for 24 hrs, 100 μL of the reaction product is transferred to separate, flat bottom 96-well plate. The absorbance of Alamar Blue is read using a microplate reader (FLUOstar Omega, BMG Labtech). Increasing absorbance indicates increasing cell viability.

### Alkaline Phosphatase Activity

The effect of 70S and 70STi glass powders in contact with HDPSCs was assessed based on alkaline phosphatase (ALP) activity. Cells are seeded into wells of a 48-well plate at 3000 cells/well and cultured for 4 days until confluent. The respective glass powders are then added (2 mg/well) and incubated at 37° C. for 10 days. The cell plates are removed from the incubator and placed on ice to cool. The media is aspirated, and the cells are washed 2-3 times with ice cold washing buffer (20 mM Tris-HCL). The washing buffer is aspirated and the ice-cold cell lysis buffer (20 mM Tris-HCL, 200 mM NaCl, 2.5 mM MgCl2, 0.05% Triton X-100) is pipetted vigorously over cells then repeatedly up and down 20 times to burst and detach the cells from the plate. The lysate is collected in 1.5 mL tubes on ice and then centrifuged at 4° C. for ten minutes at 15,000×g. The resulting supernatant is used to measure ALP activity with the LabAssay ALP KIt (Wako Chemicals).

### Enamel Disc Experiments

Samples are polished with 350, 600, 1200, and finally 2000 grit sand paper to create a smooth sample surface. Sample pucks are then put in contact with Tropicana orange juice (pH 3.5) for one hour, with magnetic stirring. Following demineralization, the samples are gently washed with DI water and kept damp until addition of bioactive glass paste. To create the paste, the bioactive glasses (70S and 70STi) are mixed with DI water in a 50/50 wt % mixture. The paste is then brushed onto enamel sample pucks with a soft Colgate children's toothbrush for 2 minutes in linear and circular brushing motions. The pucks are then stored in SBF (150 mL per puck) at 37° C. on a shaking plate for 7 days. SBF is refreshed every 3 days.

### EXAMPLE II

### Sol-Gel Bioactive Glass Composition with High TiO₂ Content

To develop titanium containing bioactive glass compositions, with TiO₂ concentration>5 mol %, and adequate bioactivity, a base catalyst was utilized during the synthesis. Basic condition increases the hydrolysis and condensation reactions, forming a highly branched polymer network with higher pore spaces distribution.

### Based Catalyzed Hydrolysis Reaction

$${HO}^{-} + \text{?} S \text{?} - O R \text{?} HO \text{?} S \text{?} - O R \text{?} \text{?} S \text{?} - OH + R OH + {O R}^{-} \\ \text{?} \text{indicates text missing or illegible when filed} \qquad ( 1 )$$

10% mole concentration of titanium was incorporated into the glass network. Table 4 below shows the glass compositions.

TABLE 4
Formulated Bioactive Glass Compositions

| SIN | Name | SiO₂ | CaO | TiO₂ | P₂O₅ |
|---|---|---|---|---|---|
| 1. | 60S(0-10)Ti | 60.00 | 30-35.00 | 5-10.00 | 0 |
| | (mol %) | | | | |
| 2. | 65S7.5Ti4P | 65.00 | 23.50 | 7.5.00 | 4.00 |
| | (mol %) | | | | |

The following reactants were used:

| | |
|---|---|
| Tetraethyl Orthosilicate (TEOS) | Si(OC₂H₅)₄ |
| Titanium Isopropoxide (TIP) | Ti(C₃H₇O)₄ |
| Ethanol (ETOH) | C₂H₅OH |
| Acetic Acid | CH₃COOH |
| DI water | H₂O |
| Calcium Hydroxide | Ca(OH)₂ |
| Calcium Nitrate Tetrahydrate | Ca(NO₃)₂•4H₂O |

Essentially, the experimental procedure followed is as described herein:

1. TEOS+ETOH

2. TEOS+ETOH+H₂O+Ca(OH)₂ (allowed to stir for 1 hr) Took pH value.

3. TEOS+ETOH+H₂O+Ca(OH)₂+Ca(NO₃)₂.4H₂O (allowed to stir till salt dissolved completely) Took pH value.

4. TIP+ETOH+Acetic Acid (left for 1 hr)

5. TEOS+ETOH+H₂O+Ca(OH)₂+Ca(NO₃)₂.4H₂O+(TIP+ETOH+Acetic Acid) (allowed to stir for 30 mins) Took pH value.

6. Adjusted the pH to 9

7. Aged at 37° for 2 days

8. Air dried for 24 hrs

9. Dried at 150° C. for 1.5 hr

10. Calcined at 700° C. for 6 hrs

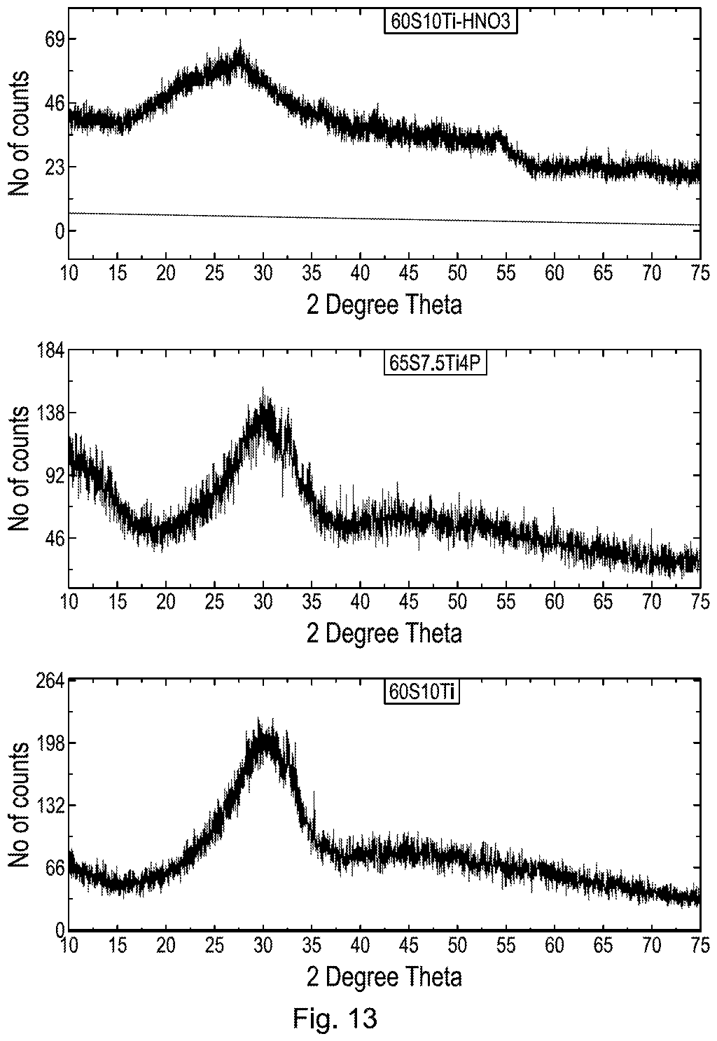

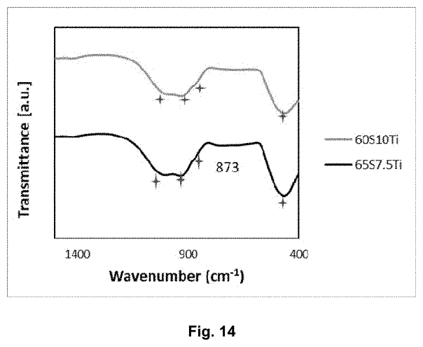

The glass samples were characterized using XRD, FTIR and XPS. As seen in FIGS. 13 and 14, samples 60S and 60S10Ti—HNO₃ are shown as control samples for comparison: 60S does not contain titanium (composition: 60 mol % SiO₂ and 40 mol % CaO); 60S10Ti—HNO₃ has the same composition as 60S10Ti but was produced using HNO₃ as a catalyst instead of CaOH₂.

As demonstrated below, from the XRD analysis, the glass samples are completely amorphous. The bands seen in FIG. 14 at 460 cm⁻¹, 873 cm⁻¹, 922 cm⁻¹ and 1000 cm⁻¹ corresponds to δ_{(SI—O—SI)}, v_{(SI—OH)}, and v_{as(SI—O—SI)} which are bands of a silica glass network.

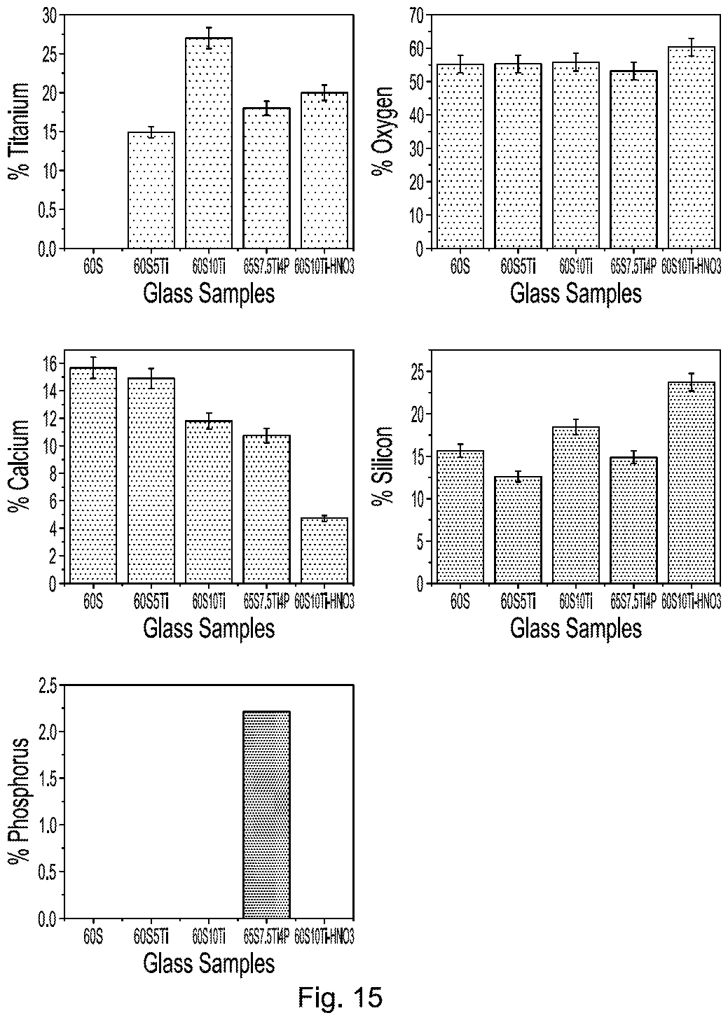

XPS shows Ti on all the Ti-containing glasses, in higher amounts on the base-catalyzed glass 60S10Ti despite the same % of Ti was added in the control, acid catalyzed glass 60S10Ti—HNO₃ (FIG. 15). A similar observation can be made for Ca. More was observed on the base-catalyzed compared to the acid catalyzed glass. P was detected only on 64S7.5Ti4P, as expected.

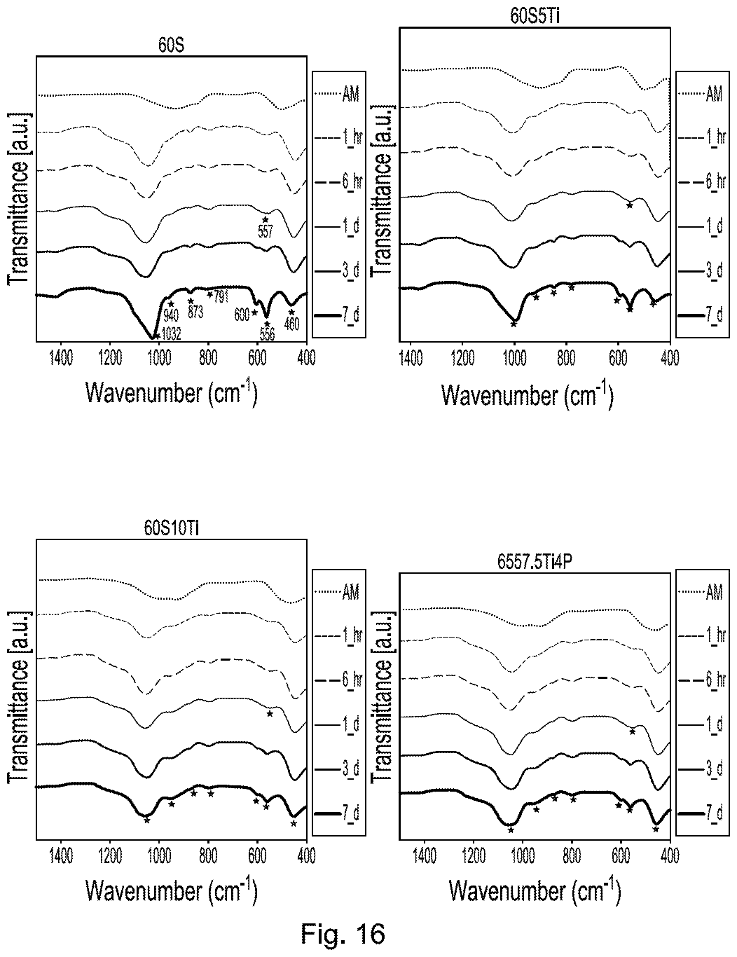

The reactivity of the bioactive glass was analyzed in simulated body fluid for 7 days, prepared following the procedure outlined by Kokubo et al. (2006, Biomaterials, 27(15): 2907-2915) (see FIG. 16). Formation of amorphous calcium phosphate can be seen after 1 hr of immersion in SBF on all the base-catalyzed glasses and on the control sample without Ti. At day 3, there is crystallization of hydroxyapatite on all glass surfaces. This result contrasts with what has been reported in literature.

While the disclosure has been described in connection with specific embodiments thereof, it will be understood that it is capable of further modifications and this application is intended to cover any variations, uses, or adaptations, including such departures from the present disclosure as come within known or customary practice within the art and as may be applied to the essential features hereinbefore set forth, and as follows in the scope of the appended claims.

What is claimed is:

1. A bioactive glass composition comprising titanium (Ti) homogenously incorporated and distributed throughout the glass, wherein at least a portion of the Ti is retained at the glass surface when the glass is in contact with a physiological solution and becomes incorporated within an hydroxyapatite layer.

2. The bioactive glass of claim 1, further comprising calcium, magnesium, sodium, phosphate, potassium, lithium, or a combination thereof.

3. The bioactive glass of claim 1, wherein said silica is in the form of tetraethyl orthosilicate (TEOS), tetramethylorthosilicate (TMOS), SiO₂, Na₂SiO₃ or a combination thereof.

4. The bioactive glass of claim 2, comprising P₂O₅, H₃PO₄, NaH₂PO₄, Na₃PO₄, CaHPO₄, Ca₃(PO₄)₂, apatite, hydroxyapatite, or a combination thereof.

5. The bioactive glass of claim 1, wherein said titanium is TiO₂.

6. The bioactive glass of claim 1, wherein said glass is a melt-derived bioactive glass or a sol-gel bioactive glass.

7. The bioactive glass of claim 1, further comprising an organic precursor.

8. The bioactive glass of claim 7, wherein said organic precursor is triethyl phosphate (TEP).

9. The bioactive glass of claim 1, wherein the glass comprises between 5 to 20 wt % of Ti.

10. The bioactive glass of claim 1, wherein the glass comprises >5 mol % of TiO₂.

11. The bioactive glass of claim 1, wherein said glass when immersed in a body fluid forms a titanium substituted hydroxyapatite (HA).

12. The bioactive glass of claim 1, wherein said glass provides enamel remineralization, enamel whitening and/or increases osteogenic differentiation.

13. A method of preparing the bioactive glass of claim 1 comprising the steps of:
a) incorporating titanium (Ti) to silica producing a mixture;
b) mixing said mixture containing titanium to high viscosity producing a sol;
c) casting the sol in a vial and gelling said sol resulting in a gel; and
d) calcine said gel at high temperature producing the titanium-containing bioactive glass.

14. The method of claim 13, wherein the silica and titanium are mixed producing a powder mixture, said powder mixture being further melted and quenched at low temperature resulting in melt-quenched titanium-containing bioactive glass.

15. The method of claim 13, further comprising incorporating calcium, magnesium, sodium, phosphate, potassium, lithium, or a combination thereof to the mixture of Ti and silica.

16. The method of claim 13, wherein the titanium is incorporated using an acid catalyzer or a basic catalyzer.

17. The method of claim 16, wherein the acid catalyzer is HCl or HNO₃, or the basic catalyzer is NaOH, Na₂CO₃, NH₄OH or CaOH₂.

18. The method of claim 16, wherein the basic catalyzer is CaOH₂.

19. The method of claim 13, wherein the glass comprises >5 mol % of TiO₂.

20. The method of claim 15, wherein the silica is in the form of tetraethyl orthosilicate (TEOS), tetramethylorthosilicate (TMOS), SiO₂, Na₂SiO₃ or a combination thereof; and wherein the calcium is in the form of CaO, CaCO₃, Ca(OH)₂, Ca(NO₃)₂, calcium oxalate, calcium acetate, or a combination thereof.
